(19) United States
(12) Patent Application Publication
(10) Pub. No.: US 2017/0172495 A1
(43) Pub. Date: Jun. 22, 2017

(54) METHOD AND SYSTEM FOR DETECTING AND IDENTIFYING DIFFERENT TYPES OF PAIN AND MONITORING SUBSEQUENT THERAPY

(71) Applicant: THE UNIVERSITY OF NEWCASTLE, Callaghan, New South Wales (AU)

(72) Inventors: Carolyn Mountford, East Ryde NSW (AU); Saadallah Ramadan, Bexley NSW (AU); Alexander Lin, Boston, MA (US)

(21) Appl. No.: 15/127,377

(22) PCT Filed: Mar. 18, 2015
(86) PCT No.: PCT/US15/21312

§ 371 Date: Sep. 19, 2016

Related U.S. Application Data
(60) Provisional application No. 61/955,048, filed on Mar. 18, 2014.

(51) Int. Cl. A61B 5/00; G01R 33/48; G01R 33/46; A61B 5/055

(52) U.S. Cl. CPC A61B 5/4824; A61B 5/055; A61B 5/4255; A61B 5/4571; A61B 5/4839; A61B 5/0042; A61B 5/742; A61B 5/4848; G01R 33/4806; G01R 33/4633

(57) ABSTRACT

The present invention relates to a method and system for using neurochemical markers to identify different types of pain, including chronic and acute, and providing the capacity to monitor response to therapy on an individual basis. The present invention relates to a method and system for using neuro biomarkers to identify pain of different types and origins, and the capacity to monitor response to therapy on a personalized basis.

### TECHNICAL FIELD OF THE INVENTION

The present invention relates to a method and system for using neurochemical markers to identify different types of pain, including chronic and acute, and providing the capacity to monitor response to therapy on an individual basis. The present invention relates to a method and system for using neuro biomarkers to identify pain of different types and origins, and the capacity to monitor response to therapy on a personalized basis.

### BACKGROUND OF THE INVENTION

This patent application cites various publications and other documents. These publications and documents are hereby incorporated by reference.

Evidence based medicine is currently used to manage chronic pain i.e. each individual's clinical management is based on the outcomes from other people. The flaw in this approach is that risk of acute and chronic pain and response to treatment varies from person to person due to differences in genetic makeup, environmental exposure and insult to the body and type of pain. Personalized medicine is now being implemented from research outcomes where innovations are designed to customize care [1]. However its success is critically dependent on the reliability and increased precision for diagnosis and monitoring therapy. There are different origins and types of chronic pain. Distinguishing between these categories by an objective means is important for the patient and the healthcare budget.

Pain is one of the most expensive health problems [2], costing the Australian economy, for example, around $34 billion every year [3]. Chronic pain significantly impairs quality of life, as manifested by poorer mental and physical function, work productivity, relationships and sleep.

### SUMMARY OF THE INVENTION

The present invention provides a system and method for using 2D COSY to detect pain. The present invention is also directed to using two dimensional (2D) neuro magnetic resonance spectroscopy (MRS) to detect neurochemical markers to identify and distinguish between from different origins. Neurochemical markers that alter with chronic pain, including neuropathic pain, nociceptive pain, chronic prostatitis/pelvic pain syndrome (CP/CPPS) and irritable bowel syndrome (IBS) can be identified by 2D MRS, and more particularly 2D COSY. An objective diagnosis can be made for each of these pain types where in some cases further subcategorization is possible. The biomarkers can improve clinical management by providing a definitive diagnosis; an understanding of the biological pathways; and for the first time a means to objectively test new therapies.

MR technology, two-dimensional (2D) MR spectroscopy, allows definitive assignment of neurochemicals that alter with pain, head injury and a range of neurological diseases.

The invention provides a system and method to apply MR technologies to document the neurochemical effects of chronic pain of different types and origins.

The MR data can be analyzed by a modern informatics and now shown to be effective for a study on chronic pelvic pain syndrome, neuropathic and noiciceptive pain. The outcome can yield informatics outcomes for automated specific molecular information on altered pathways for the development of improved pharmacologic intervention; the capacity to monitor therapy; and tools for clinical assessment of recovery.

How chronic pain alters brain chemistry can be monitored by neuromagnetic resonance spectroscopy (MRS). In contrast to morphological magnetic resonance imaging (MM), or functional MRI (fMRI) which characterizes temporal differences in brain activity in response to stimulation, MRS monitors changes in the chemical activity in the brain. It is suggested [4] that neuro MRS identifies the earliest changes to the brain. Neuro MRS can be correlated with modern techniques such as diffusion tensor imaging (DTI) and susceptibility weighted imaging (SWI).

Clinically there is significant overlap between chronic pain from conditions such as irritable bowel and other poorly understood chronic pain condition syndromes including CP/CPPS [5]. These are clinically distinguishable from the more established neuropathic and noiciceptive types of chronic pain. One can use one-dimensional (1D) MRS, two-dimensional (2D) COrrelated SpectroscopY (COSY) and separate neuropathic from Noiciceptive from CP/CPPS cohort from the controls (p<0.01). The diagnostic spectral regions identified by 2D MRS can be assigned to free and bound fucose, and glutamate and glutamate as well as other markers such as choline. The terminal fucoslyated molecules are understood to be associated with the inflammatory process as well as normal brain activities. The level of pain can be determined by analysis of the MRS data. In contrast to neurochemical changes from neuropathic and noiceptive pain [6-8] which includes glutamatergic dysfunction, CP/CPPS does not exhibit glutamatergic dysfunction and can be subcategorized.

### DESCRIPTION OF THE DRAWINGS

FIG. 1 is a 1D MRS of spinal cord injury-without pain, compared with spinal cord injury-with pain patient, with and without pain [8];

FIG. 2 shows:
A. 1D & 2D COSY from the PCG of CP/CPPS patients at 3T (12 channel coil) and 3×3×3 cm³ voxel. Assignments see [9]. Right hand side, the expansion of F2: 4.0-4.5, F1: 1.1-1.7);
B. Healthy control; and
C. CP/CPPS. This spectral region is increased (P<0.05) by 21% in patients with CP/CPPS (0.011±0.003) and in healthy controls (0.009±0.001). Based on assignments CP/CPPS divided into three groups: presence of either Fuc II or IV or non. The IBS case had Fuc II ie a marker of neuropathic pain: and

FIG. 3 shows a system for practicing the invention.

### DESCRIPTION OF A PREFERRED EMBODIMENT

A preferred embodiment will be described as one way of practicing the invention, but the invention is not limited to this embodiment.

Using 2D MRS data, one can assign the diagnostic molecules and identify the neurochemical pathways that alter with each type of pain, such as chronic pain and its various types.

The chemicals that alter the neurochemical pathways reflect specific pathways that have been altered as a function of the origin of the chronic pain.

One can detect several different subcategories of chronic pain: those with glutamatergic dysfunction and those without. For example neuropathic and nociceptive pain will include glutamatergic dysfunction as well as fucosylated markers. IBS and CP/CPPS will not have glutamatergic dysfunction but will have fucosylated markers. There will be at least two different types of fucoslyated markers distinguishing neuropathic from other types of pain.

Central sensitization, contributes to inflammatory, neuropathic, and functional pain [10]. This hypersensitivity arises when the pain pathways increase in sensitivity when relaying pain messages. It is suggested that fibromyalgia and IBS could be manifestations of altered function of the nervous system involving central sensitization [10]. Deregulation of the acute tryptophan depletion brain network is thought to promote central pain amplification in IBS [11] which is difficult to correct A biomarker(s) of central sensitization [12] would be important. Biomarkers for inflammation have been identified and their role in the amplification process can thus be considered.

Early in vivo spectroscopy studies undertaken in neuropathic pain patients reported a reduction in NAA and glucose in the prefrontal cortex (PFC) in chronic low back pain sufferers [13] and reduction of NAA in the thalamus [14]. More recent studies, with improved MRS technology, on chronic low back pain and migraine cohorts [6-8] have shown glutamatergic dysfunction known to cause neuronal damage above certain levels [15]. A personalized medicine approach will in the future provide an understanding of pain biology.

Described as a “virtual biopsy”, MRS obtains chemical information from specific regions of interest (ROI or voxel). Neuro metabolites relevant to chronic pain, diseases, degeneration and psychological categories can be measured relative to each other [8]:
NAA (n-acetyl aspartate): is a marker of viable neurons, axons and dendrites.
Glx (combination of glutamate and glutamine): Glutamate is the primary excitatory neurotransmitter in the brain and is tightly coupled to glutamine which is found in the astrocytes.
Cr (creatine): is frequently used as an internal standard.
Cho (choline): is a membrane marker which increases with pathological alterations.
mI (myo-inositol): is an astrocyte marker and osmolyte and increases with membrane damage.
Lipid: lipid not “MR visible” unless liberated by a severe a pathological process.
Lactate: Lactate in the brain reveals aging process [16].
Phenylalanine (Phe): Increases with repetitive head injury and is indicative of a breakdown in the tyrosine kinase pathway which in turn affects dopamine levels and causes depression.
Fucose (Fuc): Fucose-α(1-2)-galactose [Fucα(1-2)Gal] implicated in the molecular mechanisms that underlie neuronal development, learning, and memory [17]. Increased and altered fucose has been linked to early events of inflammation [18, 19] [20, 21].

With sufficient numbers in each category the 1D MRS method can be analyzed by robust mathematical methods removing the need for a reader.

In the 1D spectrum many resonances are composites and/or overlap. Two dimensional (2D) MRS, COrrelated SpectroscopY (COSY), can be employed in vivo to separate resonances in a second magnetic frequency (FIG. 2) [9, 22]. For a list of assignments see Ramadan [9]. While it is possible to use 1D spectral editing technique to identify metabolites that overlap, the advantage of the 2D method is that the second frequency reveals all chemical species within a single exam and their ratios are directly comparable. Many more molecules are available for inspection but importantly the cross peaks that report on the scalar coupling provide a more accurate measurement [9] of changes that are occurring from disease, impact, degeneration in response to chronic pain [15].

Our recent studies have shown that the neurochemical changes from neuropathic and noiceptive pain i.e. chronic low back pain [7], chronic pain from spinal cord injury [8] and migraine [6] have some commonalty and some differences.

The 2D COSY method confirmed glutamatergic dysfunction i.e. increased levels of glutamate/glutamine in chronic noiceptive (osteoarthritis) and neuropathic (postherpetic neuralgia) pain but have yet to confirm if glutamine and or glutamate, or both, that are increasing. Glutamate, an excitatory neurotransmitter, is predictive of poor outcome when elevated in severe traumatic brain injury [23]. The concept of the excitory amino acids, particularly glutamate, having a role in neurologic disorders has been discussed [24]. Other differences include increases in choline and myoinositol.

The assignment of fucosylated molecules in the brain was made possible by the 2D method and modern MR scanner technology [9]. Four fucosylated species have been assigned [25] in cancer cell models and in vivo in glioma [9]. Finding similar species in 2D spectra from neuropathic pain patient was unexpected. Neuropathic pain has Fuc IV [9] present in addition to Fuc I and III whereas the noiceptive cases have Fuc II as well as Fuc I and III. Two cases of TN have been examined and increased glutamatergic dysfunction recorded as well as the appearance of Fuc IV. Fucose I and III were recorded in the control cohort but not Fuc II or Fuc IV. Free fucose was also recorded in most chronic pain cases.

CP/CPPS and IBS are often accompanied by “associated negative cognitive, behavioral or emotional” consequences. Fucose-α(1-2)-galactose [Fucα(1-2)Gal] sugars are implicated in the mechanisms that underlie neuronal development, learning, and memory [17]. The increase in free fucose and in the fucosylated epitopes associated in the early events of inflammation [18, 19] may affect the equilibrium and thus behavior and mental capacity. [18, 19] [20, 21].

Chronic prostatitis/chronic pelvic pain syndrome as used herein is defined as “urologic pain or discomfort in the pelvic region, associated with urinary symptoms and/or sexual dysfunction, lasting for at least 3 of the previous 6 months” in the absence of any identifiable pathology such as cancer, culturable infection, or anatomic abnormalities, often accompanied by “associated negative cognitive, behavioral, sexual or emotional consequences.

Neurochemical changes were found to occur in the brain of patients with CP/CPPS (FIG. 2). The 1D MRS data found differences between the healthy cohort and those with CP/CPPS with a significance of p<0.01 for the ACC and PCG. This is the first time biomarkers have been identified for CP/CPPS. The MRS method also reported on the level of pain in comparison to the clinical records.

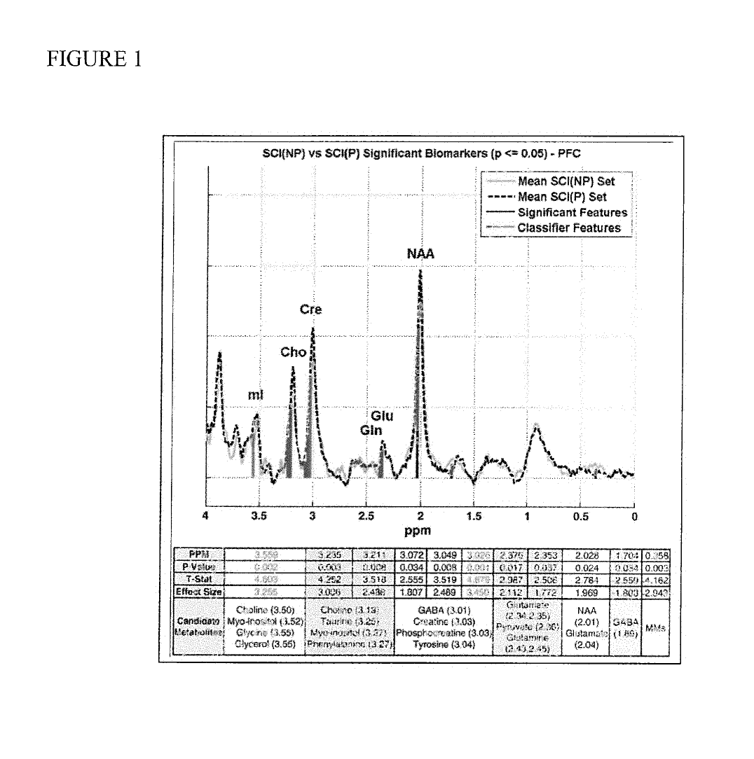

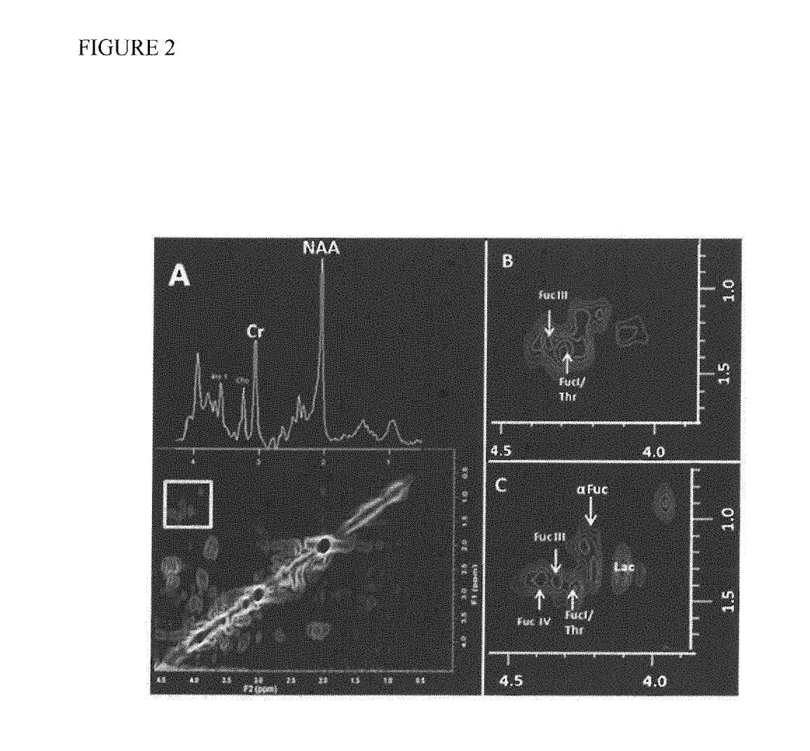

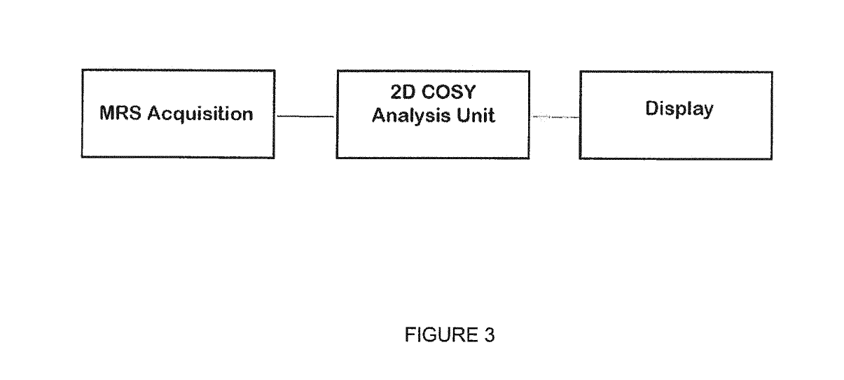

The 2D COSY method identified the fucose region as being diagnostic for CP/CPPS. This spectral region, is significantly increased (P<0.05) by 21% averaged over patients with CP/CPPS (FIGS. 1 B & C). The altered spectral regions were assigned to the two unique fucose markers Fuc II and Fuc IV [25] linked to inflammation[18, 19] [20, 21] and an increase in free fucose neither of which are present in the healthy cohort. When compared with cases of TN (neuropathic) and migraine (noiceptive) and the CP/CPPS cohort divided into two categories viz. normal; comparable with purely neuropathic pain; and comparable with noiceptive pain. There were no differences recorded in NAA, Glx, choline or mI with CP/CPPS in this region of the brain. Thus CPPS and the one case of irritable bowel syndrome in this cohort did not demonstrate glutamatergic dysfunction which make them different to neuropathic pain, noiceptive pain and mixed pain from spinal cord injury.

One can use a Siemens research clinical scanner, the PRISMA. The system has a 64 channel head and neck coil. We showed during a repetitive head injury study that the 32 channel gave reproducibility to 3% in contrast to the 12 channel of 8%.

In summary, neuro 2D MRS monitors alterations to neurochemical pathways associated with chronic pain and inflammation. One can determine the effects of upward (sensory neural connections) resulting in chronic pain and inflammation; and downward (motor and regulatory) neural connections on gut function.

One can collect 1D MRS data and analyze using the modern informatics to generate objective diagnostic tests for chronic pain from different origins.

Using 2D MRS data one can assign the diagnostic molecules and identify the neurochemical pathways that alter with each type of chronic pain.

One can also monitor the effect of therapy on patients with chronic pain. The spectral information can be analyzed and compared with the clinical outcomes. The effects of therapy and levels of pain can also be analyzed by modern informatics such as that discussed in U.S. Pat. No. 6,835,572 or U.S. Pat. No. 7,676,254.

All MR data can be acquired on a Siemens 3T Prisma using a 64 channel head and neck coil. The Prisma has double the current gradient strength (80 milli tesla per meter (mT/m) and a slew rate of 200 tesla per meter per second (T/m/s)), high order shims. The VD13D operating software incorporates the automated shim routine and capacity to control the water suppression [26].

Before undergoing spectroscopy, patients can be imaged with an MR imaging protocol that includes T2-weighted three-dimensional turbo spin-echo imaging with variable flip angle (repetition time=3.2 seconds, echo time=494 msec, section thickness=0.9 mm). Localized shimming can be performed with automatic adjustment of first- and second order shim gradients by using the automatic three-dimensional B 0 field mapping technique (Siemens) to achieve a magnitude peak width of water at half maximum resonance height of 14 Hz or less. After frequency adjustment, water-selective suppression can optimized by the VD13D software.

Single voxel short echo (TR/TE: 1500/30 ms, PRESS, voxel size: 2×2×2 cm³, 96 averages) can be acquired in; 1) prefrontal cortex; 2) parietal white matter, and 3) anterior white matter. Scan time can be 5 minutes per voxel.

A two-dimensional Correlated SpectroscopY (2D COSY) can be acquired in chosen brain regions with the following parameters: RF carrier frequency at 2.0 ppm; TR 1.5 s; weak water suppression using WET; spectral width of 2000 Hz; increments size of 0.8 ms in 64 t1 increments giving an indirect spectral width of 1250 Hz; 8 averages per increment; and 1024 data points. Scan time for the 2D COSY can be 11 minutes.

Structural imaging can be obtained with T1-weighted MPRAGE volumetric sequence (TR/TE=2530/1.7 ms, 12 degree flip angle, FOV=256×256 mm, voxel size 1×1×1 mm, NEX 4, acquisition time 6 minutes).

Diffusion tensor imaging (DTI) can consist of a 35-direction scan (TR/TE=9880/88 ms; FOV=256×256 mm; 2 mm slice thickness with 2×2 mm² in-plane resolution; b-value=0 and 1000 s/mm2) for a scan time of 6 minutes.

Susceptibility Weighted Imaging (SWI) data can be acquired using the following parameters: TR/TE=28/20 ms, FOV 256×256 mm, 1 mm slice thickness with 1×1 mm² in-plane resolution for a scan time of 6 minutes.

Quality control (Q/C) for MM and MRS data can be maintained by weekly phantom scans using both imaging and spectroscopy-specific phantoms.

In place of 2D COSY analysis, the 1D raw spectra of COSY can be concatenated into a two-dimensional array using Matlab. A Felix-2007 package (Accelrys, San Diego, Calif., USA) can be used for spectral processing and analysis[9]. The in vivo 2D spectra can be referenced to the prominent singlet diagonal peak of creatine Cr (F2=F1=3.02 ppm). Crosspeak and diagonal volumes can be measured as described in Lean et al Biochemistry. Felix-2007 processing software can provide the peak volumes of metabolite ratios in reference to Cr.

Statistical t-tests can be used to compare IBS groups and controls. In addition, multiANOVA statistical tests can be conducted to examine relationships between the MRI/MRS metrics and the PCSS, ImPACT, and balance error test scores. At follow-up, mean of groups as well as ratio of second to first scan for each individual can be compared.

The documentation of specific biochemical changes e.g., increases in excitatory amino acids; fucose (inflammation) can provide targets for pharmacological interventions going forward.

A preferred embodiment has been disclosed as one way of practicing the invention, but the invention is not limited to this embodiment. The scope of the invention is determined only by way of the following claims.

### REFERENCES CITED, INCORPORATED BY REFERENCE

1. Weston A D and Hood L, Systems Biology, Proteomics, and the Future of Health Care: Toward Predictive, Preventative, and Personalized Medicine. J Proteome Res, 2004. 3(2): p. 179-196.
2. Cousins, M. (2012) http://www.painaustralia.org.au/about-us/who-we-are.html. Pain Australia.
3. The high price of pain: the economic impact of persistent pain in Australia November 2007 2007, MBF Foundation in collaboration with University of Sydney Pain Management Research Institute.
4. Borsook, D., et al., Neuroimaging revolutionizes therapeutic approaches to chronic pain. Mol Pain, 2007. 3: p. 25.
5. Rodriguez, M. A., N. Afari, and D. S. Buchwald, Evidence for overlap between urological and nonurological unexplained clinical conditions. J Urol, 2009. 182(5): p. 2123-31.
6. Prescot A, et al., Excitatory neurotransmitters in brain regions in interictal migraine patients. Molecular Pain, 2009. 5(34).
7. Siddall, P. J., et al., Magnetic resonance spectroscopy detects biochemical changes in the brain associated with chronic low backpain: A preliminary report. Anesthesia and Analgesia, 2006. 102(4): p. 1164-1168.
8. Stanwell P, et al., Neuro magnetic resonance spectroscopy using wavelet decomposition and statistical testing identifies biochemical changes in people with spinal cord injury and pain. NeuroImage, 2010. 53.(2): p. 544-52.
9. Ramadan S, et al., In Vivo Two Dimensional MR Spectroscopy Compares the Biochemistry of the Human Brain and Glioblastoma. Radiology, 2011. 259(2): p. 540-9.
10. Wolf C J, Pain: Moving from Symptom Control toward Mechanism-Specific Pharmacologic Management. Annals of Internal Medicine PHYSIOLOGY IN MEDICINE: A SERIES OF ARTICLES LINKING MEDICINE WITH SCIENCE Physiology in Medicine, 2004. 140: p. 441-451.
11. Labus J S, et al., Acute tryptophan depletion alters the effective connectivity of emotional arousal circuitry during visceral stimuli in healthy women. Gut, 2011. 60(9): p. 1196-203.
12. Wolf C J, Central sensitization: implications for the diagnosis and treatment of pain. Pain, 2011. 152(S2-15).
13. Grachev, I.D., B. E. Fredrickson, and A. V. Apkarian, Abnormal brain chemistry in chronic back pain: an in vivo proton magnetic resonance spectroscopy study. Pain, 2000. 89(1): p. 7-18.
14. Fukui, S., Matsuno, M., Inubushi, T., Nosaka, S., N-Acetylaspartate concentrations in the thalami of neuropathic pain patients and healthy comparison subjects measured with 1H-MRS. Magn. Reson. Imaging, 2006. 24: p. 75-79.
15. Carder, R. K. and S. H. Hendry, Neuronal characterization, compartmental distribution, and activity-dependent regulation of glutamate immunoreactivity in adult monkey striate cortex. J. Neurosci, 1994. 14: p. 242-262.
16. Ross, J. M., et al., High brain lactate is a hallmark of aging and caused by a shift in the lactate dehydrogenase A/B ratio. Proceedings of the National Academy of Sciences, 2010.
17. Murrey H E, et al., Identification of the Plasticity-Relevant Fucose-α(1-2)-Galactose Proteome from the Mouse Olfactory Bulb. Biochemistry, 2009. 48(30): p. 7261-7270.
18. De Graaf T W, et al., Inflammation-induced expression of sialyl Lewis X-containing glycan structures on alpha 1-acid glycoprotein (orosomucoid) in human sera. J Experimental Med, 1993. 177(3): p. 657-666.
19. Smalla, K. H., et al., Identification of fucose-alpha[1-2]-galactose epitope-containing glycoproteins from rat hippocampus. Neuroreport, 1998. 9: p. 813-817.
20. Hoeche N, et al. Mapping fucosylatod synaptic proteins. in FENS Forum 2010. Amsterdam.
21. Lowe J B, Glycan-dependent leukocyte adhesion and recruitment in inflammation. Curr Opin Cell Biol, 2003. 15: p. 531-538.
22. Ramadan, S. and C. Mountford, Two Dimensional Magnetic Resonance Spectroscopy on Biopsy and In Vivo, in Annual Reviews in NMR, G. Webb, Editor. 2009, Academic Press.
23. Chamoun, R., et al., Role of extracellular glutamate measured by cerebral microdialysis in severe traumatic brain injury. J Neurosurg, 2010.
24. Lipton S A and RosenbergPA, Excitatory Amino Acids as a Final Common Pathway for Neurologic Disorders. N Engl J Med, 1994. 330: p. 613-622.
25. Lean, C. L., et al., Cell-surface fucosylation and magnetic resonance spectroscopy characterization of human malignant colorectal cells. Biochemistry, 1992. 31(45): p. 11095-105.
26. Mountford, C. E., et al., MR spectroscopy in the breast clinic is improving. European Journal of Radiology, 2012. 81: p. S104-S106.

1. A method for detecting whether a subject is experiencing pain, comprising:
obtaining MR spectral data from a subject's brain tissue using a MR spectroscopy device; and
producing, from the MR spectra obtained, spectral data using 2D COSY which enables the detection of whether the subject is experiencing pain by detecting the presence of at least one marker neurochemical.

2. The method of claim 1, wherein the pain being detected is chronic pain.

3. The method of claim 1, wherein the pain being detected is identified as at least one of neuropathic pain, noicieptive pain, chronic prostatitis/chronic pelvic pain syndrome (CP/CPPS) and IBS.

4. (canceled)

5. The method of claim 1, wherein the pain being detected is a subcategory of chronic prostatitis/chronic pelvic pain syndrome (CP/CPPS).

6. The method of claim 1, wherein the pain being detected is irritable bowel syndrome (IBS).

7. The method of claim 1, wherein the pain being detected is irritable bowel syndrome (IBS), and further detecting whether the origin of IBS is infectious or non-infectious.

8. The method of claim 1, further including selecting a pain treatment plan based on the type of pain detected, and treating the subject with the selected treatment plan.

9. The method of claim 1, wherein the pain being detected is different to that recorded as a result of a head injury.

10. The method of claim 1, further including the step of treating the subject with a pain medicine, and then detecting whether the pain medicine was effective by repeating the obtaining and producing steps.

11. The method of claim 1, wherein pain is detected by detecting altered neurochemicals.

12. A method for using neurochemical markers to identify diarrhea-predominant irritable bowel syndrome of infectious from non-infectious origins in a subject, comprising:
obtaining MR spectral data from a subject's brain tissue using a MR spectroscopy device; and
producing from the MR spectra a spectral data display having at least one neurochemical marker to enable a determination of whether the subject has irritable bowel syndrome of infectious type, as compared to irritable bowel syndrome of non-infectious type.

13. The method of claim 10, wherein the spectral data is obtained and the spectral data is displayed using a magnetic resonance spectroscopy device using a 2D COSY.

14. The method according to claim 10, wherein the spectral data enables a determination of whether the subject has chronic trigeminal neuralgia.

15. The method of claim 10, wherein the spectral data is obtained from a subject's brain tissue in vivo.

16. A method for using neurochemical markers to determine whether a subject having irritable bowel syndrome is of gut-to-brain origin, as compared to of brain-to-gut origin, comprising:
obtaining MR spectral data from a subject's brain tissue using a MR spectroscopy device; and
producing from the MR spectra a spectral data display having at least one neurochemical marker to enable a determination of whether the origin of the irritable bowel syndrome is gut-to-brain type, as compared to brain-to-gut type.

17. The method of claim 14, wherein the spectral data is obtained and the spectral data is displayed using a magnetic resonance spectroscopy device using a 2D COSY.

18. The method of claim 14, wherein the spectral image enables a determination of whether the subject has chronic trigeminal neuralgia.

19. The method of claim 14, wherein the spectral data is obtained from a subject's brain tissue in vivo.

20. A system for using neurochemical markers to identify diarrhea-predominant irritable bowel syndrome of infectious from non-infectious origins in a subject, comprising:
a magnetic spectroscopy device for obtaining MR spectral data from a subject's brain tissue; and
a display for displaying spectral data having at least one neurochemical marker to enable a determination of whether the subject has irritable bowel syndrome of infectious type, as compared to irritable bowel syndrome of non-infectious type.

21. The system of claim 20, wherein the spectral data is obtained and the spectral data is displayed using a 2D COSY.

22. The system according to claim 20, wherein the spectral image enables a determination of whether the subject has chronic trigeminal neuralgia.

23. The system according to claim 20, wherein the spectral data is obtained from a subject's brain tissue in vivo.

24. A system for using neurochemical markers to determine whether a subject having irritable bowel syndrome is of gut-to-brain origin, as compared to of brain-to-gut origin, comprising:
a magnetic spectroscopy device for obtaining MR spectral data from a subject's brain tissue; and
a display for displaying spectral data having at least one neurochemical marker to enable a determination of whether the origin of the irritable bowel syndrome is gut-to-brain type, as compared to brain-to-gut type.

25. The system of claim 24, wherein the spectral data is obtained and the spectral data is displayed using a 2D COSY.

26. The system of claim 24, wherein the spectral data enables a determination of whether the subject has chronic trigeminal neuralgia.

27. The system of claim 24, wherein the spectral data is obtained from a subject's brain tissue in vivo.

28. A system for detecting whether a subject is experiencing pain, comprising:
a magnetic spectroscopy device for obtaining MR spectral data from a subject's brain tissue; and
a processor for producing, from the MR spectra obtained, spectral data using 2D COSY which enables the detection of whether the subject is experiencing pain by detecting the presence of at least one marker neurochemical.

29. The system of claim 26, wherein the pain being detected is chronic pain.

30. The system of claim 26, wherein the pain being detected is at least one of neuropathic and noicieptive pain.

31. The system of claim 26, wherein the pain being detected is chronic prostatitis/chronic pelvic pain syndrome (CP/CPPS).

32. The system of claim 26, wherein the pain being detected is irritable bowel syndrome (IBS).

33. The system of claim 26, wherein the pain being detected is irritable bowel syndrome (IBS), and whether the origin of IBS is infectious or non-infectious.

34. The system of claim 26, wherein the pain being detected is as a result of a head injury.
